(19) United States
(12) Patent Application Publication
(10) Pub. No.: US 2020/0065439 A1
(43) Pub. Date: Feb. 27, 2020

(54) SIMULATING MATERIALS USING QUANTUM COMPUTATION

(71) Applicant: Google LLC, Mountain View, CA (US)

(72) Inventors: Ryan Babbush, Venice, CA (US); Hartmut Neven, Malibu, CA (US)

(21) Appl. No.: 16/344,112

(22) PCT Filed: Nov. 10, 2016
(86) PCT No.: PCT/US2016/061386

§ 371 Date: Apr. 23, 2019

Related U.S. Application Data
(60) Provisional application No. 62/412,084, filed on Oct. 24, 2016.

(51) Int. Cl. G06F 17/50; G06N 10/00; G06F 9/448; G16C 10/00

(52) U.S. Cl. CPC G06F 17/5009; G06N 10/00; G06F 2217/16; G16C 10/00; G06F 9/448

(57) ABSTRACT

Methods, systems, and apparatus for quantum simulation of materials. In one aspect, a method includes the actions of determining a physical system of interest, wherein the physical system comprises a plurality of unit cells; performing a quantum computation to approximate a ground state of the physical system in a region of one of the unit cells; and providing the approximated ground state of the physical system in the region of the unit cell as output.

### BACKGROUND

This specification relates to quantum computing.

Quantum computers have the potential to solve certain problems faster than any classical computers that use the best currently known algorithms. In addition, quantum computers promise to efficiently solve important problems that are not practically feasible on classical computers. An example of such an important problem is calculating the eigenvalues of quantum operators, since the dimension of quantum systems grows exponentially. Determining eigenvalues of quantum operators is a core task of many practical applications of quantum computing.

### SUMMARY

This specification describes technologies for simulating materials or other physical systems of interest using a quantum computer.

In general, one innovative aspect of the subject matter described in this specification can be implemented in a method that includes determining a physical system of interest, wherein the physical system comprises a plurality of unit cells; performing a quantum computation to approximate a ground state of the physical system in a region of one of the unit cells; and providing the approximated ground state of the physical system in the region of the unit cell as output.

Other implementations of this aspect include corresponding computer systems, apparatus, and computer programs recorded on one or more computer storage devices, each configured to perform the actions of the methods. A system of one or more computers can be configured to perform particular operations or actions by virtue of having software, firmware, hardware, or a combination thereof installed on the system that in operation causes or cause the system to perform the actions. One or more computer programs can be configured to perform particular operations or actions by virtue of including instructions that, when executed by data processing apparatus, cause the apparatus to perform the actions.

The foregoing and other implementations can each optionally include one or more of the following features, alone or in combination. In some implementations the quantum computation to approximate the ground state of the physical system in the region of the unit cell comprises: defining an initial ground state of the physical system in the region of the unit cell as the ground state of a Hamiltonian for the unit cell; and iteratively processing the initial ground state and subsequent ground states until completion of an event occurs, wherein for each iteration a quantum computation is performed.

In some implementations the processing comprises for each iteration: determining an embedding Hamiltonian for the iteration; performing a quantum computation to determine a ground state of the embedding Hamiltonian for the iteration; determining whether the completion event occurs; in response to determining that the completion event has not occurred, providing the determined ground state of the embedding Hamiltonian for the iteration as a subsequent state; and in response to determining that the completion event has occurred, defining the determined ground state of the embedding Hamiltonian as an approximated ground state of the physical system in the region of the unit cell.

In some implementations determining an embedding Hamiltonian for the iteration comprises performing a classical computation.

In some implementations performing the classical computation comprises applying Density Matrix Embedding Theory (DMET).

In some implementations performing the quantum computation to determine the ground state of the embedding Hamiltonian for the iteration comprises performing a variational method.

In some implementations the variational method comprises a variational quantum eigensolver.

In some implementations performing the variational method comprises performing one or more quantum computations and one or more classical computations.

In some implementations the completion of the event occurs when a processed ground state for the iteration converges with a processed ground state for the previous iteration.

In some implementations the approximated ground state of the physical system in the region of the unit cell describes properties of the whole physical system.

In some implementations a unit cell defines a symmetry and structure of the physical system.

In some implementations the physical system is a material.

In some implementations the method further comprises using the outputted ground state of the physical system in the region of the unit cell to simulate properties of the material.

In some implementations the method further comprises using the outputted ground state of the physical system in the region of the unit cell to determine properties of the physical system.

The subject matter described in this specification can be implemented in particular ways so as to realize one or more of the following advantages.

A system simulating materials, e.g., properties of materials, using quantum computation may be used to simulate physical systems, described by finite Hamiltonians, in the presence of a correlated environment. Furthermore, quantum computers can exactly simulate physical systems in time that is at most polynomial in system size. Therefore, unlike other systems, a system simulating materials using quantum computation is not fundamentally limited by the accuracy of classical calculations used in the simulation process. For example, classical techniques for simulating physical systems, such as Density Matrix Embedding Theory (DMET), Density Matrix Renormalization Group, Hartree-Fock, or Coupled Cluster, can only obtain target accuracy in modeling physical systems at a cost which is exponential in the physical system size.

Such classical methods can therefore only accurately model certain physical systems, e.g., those that are composed of relatively small unit cells or that exhibit low amounts of correlation. A system simulating materials using quantum computation, however, extends the reach of classical methods and may be used to simulate a wide variety of physical systems—including those exhibiting strong amounts of correlation. Example physical systems include materials, e.g., polymers in airplane wings and rockets, solar cells, batteries, catalytic converts or thin film electronics. Other example physical systems include systems exhibiting high temperature superconductivity.

The details of one or more implementations of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 depicts an example system for simulating physical systems using quantum computation.

FIG. 2 is a flow diagram of an example process for simulating a physical system.

FIG. 3 is a flow diagram of an example process for approximating a ground state of a physical system in a region of a unit cell.

FIG. 4 is a flow diagram of an example iteration of processing a ground state to approximate a ground state of a physical system in a region of a unit cell.

Like reference numbers and designations in the various drawings indicate like elements.

### DETAILED DESCRIPTION

An apparatus and methods for simulating physical systems using quantum computation is described. The apparatus and methods model bulk properties of the physical system by modeling a region around a unit cell of the physical system using techniques for embedding Hamiltonians, e.g., Density Matrix Embedding Theory (DMET).

Typically, DMET techniques use classical methods to find a ground state of an embedding Hamiltonian, such as density matrix renormalization group, Hartree-Fock, coupled cluster or full configuration interaction. Such classical methods can only obtain target accuracy in modeling physical systems at a cost that is exponential in the physical system size. This specification describes techniques for combining classical computations, e.g., those based on DMET, with quantum computation to generate a hybrid quantum-classical method for simulating physical systems. The hybrid quantum-classical method enables general physical systems to be simulated, e.g., physical systems described by finite Hamiltonians with strong correlations.

### Example Operating Environment

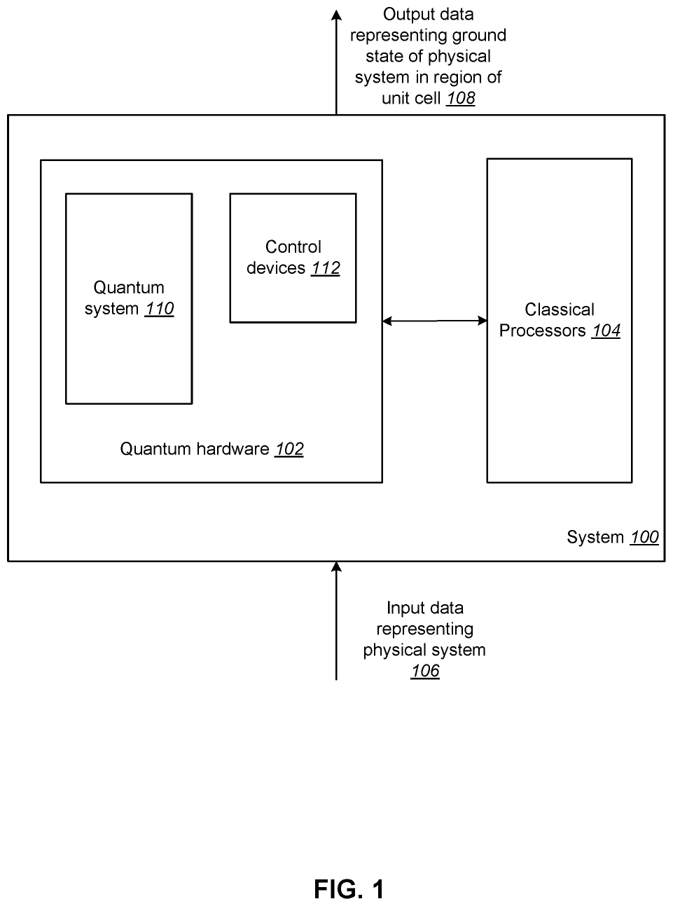

FIG. 1 depicts an example system 100 for simulating physical systems using quantum computation. The example system 100 is an example of a system implemented as classical or quantum computer programs on one or more classical computers or quantum computing devices in one or more locations, in which the systems, components, and techniques described below can be implemented.

The system 100 may include quantum hardware 102 in data communication with a classical processor 104. The system 100 may receive as input data that may include data representing a physical system of interest, e.g., input data 106. The system 100 may generate as output data for simulating the physical system of interest, e.g., output data 108.

The received data representing a physical system of interest, e.g., input data 106, may include data representing a physical system that is to be modeled or simulated. In some implementations the received data may represent a physical system that is a material, e.g., a metal or polymer. In some implementations the received data may represent a physical system describing high temperature superconductivity. The physical system represented by the received data may include multiple unit cells. A unit cell represents a smallest group of components in the physical system that constitute a repeating pattern in the physical system. Therefore, a unit cell defines a symmetry and structure of the entire physical system.

The generated data for simulating the physical system of interest, e.g., output data 108 representing the ground state of the physical system in a region of a unit cell, may include data that may be used to determine properties of the physical system. Due to the structure of the physical system, as described above, an approximated ground state of the physical system in the region of the unit cell describes properties of the physical system as a whole. Therefore, the outputted data representing the ground state of the physical system in a region of a unit cell may be used to describe properties of the entire physical system. For example, as described above, in some implementations the physical system may be a material, e.g., metal. In these cases data representing the ground state of the physical system in a region of a unit cell may be used to determine properties of the metal, e.g., conductivity.

Generating data for simulating the physical system of interest, e.g., output data 108 representing the ground state of the physical system in a region of a unit cell, may include performing quantum computation. For example, in some implementations data representing the ground state of the physical system in a region of a unit cell, e.g., output data 108, may be generated using methods that include both quantum computations and classical computations.

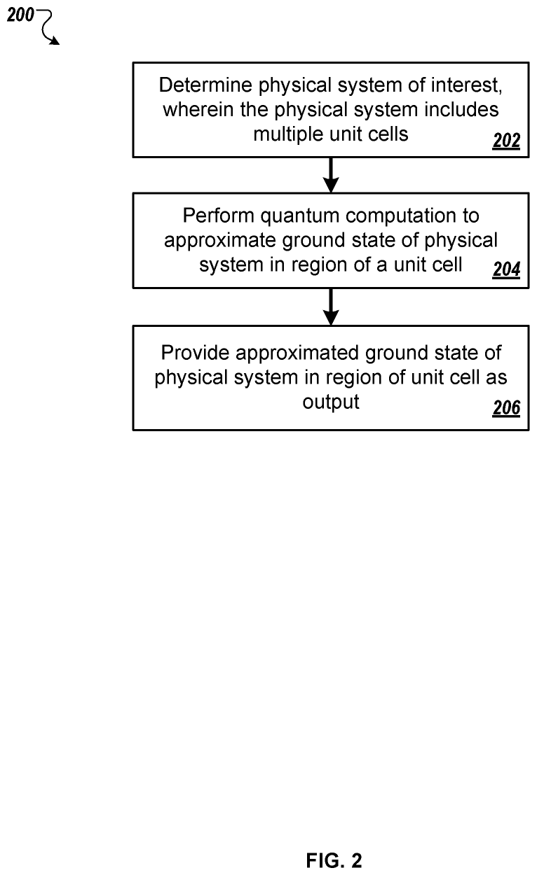

The system 100 may be configured to perform classical computations in combination with quantum computations using quantum hardware 102 and classical processors 104. In some implementations the classical processors 104 may be configured to perform techniques based on Density Matrix Embedding Theory to assist the system 100 in modeling bulk properties of the physical system of interest by only modeling a region around a unit cell of the physical system, as described below with reference to FIGS. 2-4.

For example, the physical system of interest may be described by a Hamiltonian H_{sys} with an associated ground state ψ_{sys}. Similarly, one of the unit cells that constitute the physical system of interest may be described by a Hamiltonian H_{cell} with an associated ground state ψ_{cell}. The system 100 may be configured to determine an embedding Hamiltonian H_{emb} whose associated ground state ψ_{emb} matches the ground state of ψ_{sys} in the local region of the unit cell using classical processors 104 and quantum hardware 102.

In some implementations a ground state of the embedding Hamiltonian may be determined using quantum computation. The system 100 may be configured to perform quantum computation using quantum hardware 102. The quantum hardware 102 may include components for performing quantum computation. For example, the quantum hardware 102 may include a quantum system 110. The quantum system 110 may include one or more multi-level quantum subsystems, e.g., qubits or qudits. In some implementations the multi-level quantum subsystems may be superconducting qubits, e.g., Gmon qubits. The type of multi-level quantum subsystems that the system 100 utilizes is dependent on the physical system of interest. For example, in some cases it may be convenient to include one or more resonators attached to one or more superconducting qubits, e.g., Gmon or Xmon qubits. In other cases ion traps, photonic devices or superconducting cavities (with which states may be prepared without requiring qubits) may be used. Further examples of realizations of multi-level quantum subsystems include fluxmon qubits, silicon quantum dots or phosphorus impurity qubits. In some cases the multi-level quantum subsystems may be a part of a quantum circuit. In this case the quantum hardware 102 may include one or more control devices 112, e.g., one or more quantum logic gates, that operate on the quantum system 110.

The quantum hardware 102 may be configured to determine a ground state for an embedding Hamiltonian using variational methods, e.g., variational quantum eigensolvers. For example, the quantum hardware 102 may include data specifying a variational ansatz that uses information about the quantum hardware 102, such as the control devices 112 and control parameters associated with the control devices 112, to determine a parameterization for the state of the quantum system 112. In some implementations the quantum hardware 102 may be directly used to parameterize the ansatz, that is the variational class of parameters that form the variational ansatz 116 may include the control parameters of the control devices 114, e.g., control parameters of one or more logic gates.

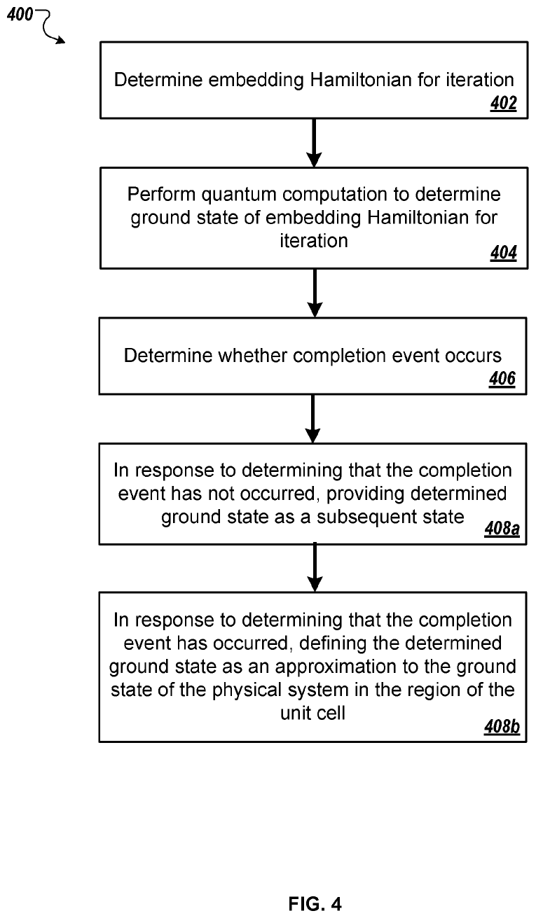

The quantum hardware 102 may be configured to perform quantum measurements on the quantum system 110 and send measurement results to the classical processors 104. In addition, the quantum hardware 102 may be configured to receive data specifying an updated parameterization for the state of the quantum system 110, e.g., updated physical control parameter values, from the classical processors 104. The quantum hardware 102 may use the received updated parameterization to update the state of the quantum system 110. Using quantum hardware to perform variational methods is described in more detail below with reference to FIG. 4.

As described above, the classical processors 104 may be configured to receive measurement results from the quantum hardware 102. The classical processors 104 may determine a minimizing parameterization for the quantum system 110 by performing a minimization method on the received measurement results, e.g., gradient-free greedy methods such as Powell's method or Nelder-Mead. In addition, the classical processors 104 may be configured to send data specifying an updated parameterization for the state of the quantum system 110 based on the determined minimizing parameterization.

Approximating the ground state of a physical system in a region of a unit cell is described in detail below with reference to FIGS. 2-4.

### Programming the Hardware

FIG. 2 is a flowchart of an example process 200 for simulating a physical system. For convenience, the process 200 will be described as being performed by a system of one or more classical or quantum computing devices located in one or more locations. For example, a quantum computation system, e.g., the system 100 for simulating materials using quantum computation 100 of FIG. 1, appropriately programmed in accordance with this specification, can perform the process 200.

The system determines a physical system of interest (step 202). The physical system of interest may be a physical system that is to be modeled or simulated. In some implementations the physical system may be a material, e.g., a metal or polymer. In some implementations the physical system may be a system exhibiting high temperature superconductivity.

The physical system includes multiple unit cells. A unit cell represents a smallest group of components in the physical system that constitute a repeating pattern in the physical system. Therefore, a unit cell defines a symmetry and structure of the entire physical system. The size of a unit cell, e.g., measured by a number of components in the unit cell, is dependent on the determined physical system. In some implementations a unit cell may interact with neighboring unit cells, and the physical system may exhibit strong correlations.

For example, some molecular systems, e.g., metals, have periodic crystal structures and are said to be “regular”. The crystal structure of a system can be described in terms of a unit cell that represents the smallest group of atoms in three dimensions that constitute a repeating pattern in the system. Stacking unit cells in three-dimensional space describe the bulk arrangement of atoms in the crystal. The unit cell can be represented in terms of one or more parameters, e.g., lattice parameters, which represent lengths of the cell's edges and angles between said edges. Positions of atoms in the unit cell may be described by a set of atomic positions measured relative to the cell's edges, e.g., lattice points.

The system performs a quantum computation to approximate a ground state of the physical system in a region of one of the unit cells (step 204). Due to the structure of the physical system, as described above with reference to step 202, an approximated ground state of the physical system in the region of one of the unit cells may be used to describe properties of the physical system as a whole. In some implementations the system may perform a quantum computation to approximate a higher level eigenstate of the physical system in a region of one of the unit cells.

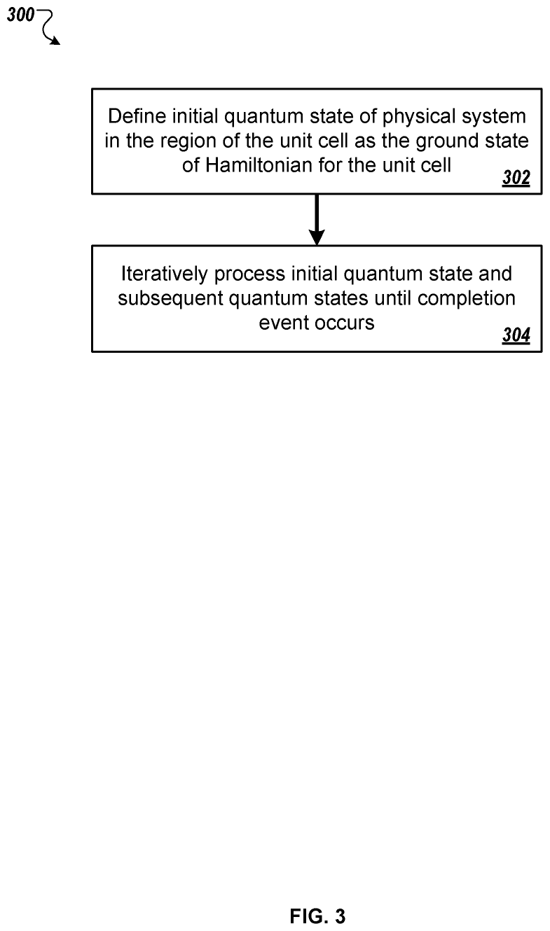

By performing a quantum computation to approximate the ground state of the physical system in a region of one of the unit cells, the system may approximate the ground state to arbitrary accuracy, e.g., where the cost of the accuracy does not scale exponentially in system size. This may enable the system to consider physical systems that are otherwise too complex to simulate, e.g., using classical methods. Performing a quantum computation to approximate a ground state of a physical system in a region of a unit cell is described in more detail below with reference to FIG. 3.

The system provides the approximated ground state of the physical system in the region of the unit cell as output (step 206). As described above, the approximated ground state of the physical system in the region of the unit cell describes properties of the physical system as a whole. Therefore, the system may use the outputted approximated ground state to simulate the physical system. For example, in some implementations the system may determine properties of the physical system using the outputted approximated ground state. In cases where the physical system of interest is a material this may include using the approximated ground state of the material in the region of a unit cell to simulate global properties of the material, e.g., using the outputted ground state to simulate the conductivity of a metal.

The process 200 can be used to simulate properties of various physical systems, including systems composed of large unit cells and/or those that exhibit strong correlations. For example, the process 200 may be used to simulate or determine properties of polymers in airplane wings and rockets, solar cells, batteries, catalytic converts or thin-film electronics.

FIG. 3 is a flowchart of an example process 300 for approximating a ground state of a physical system in a region of a unit cell. For example, the process 300 may describe approximating a ground state of a physical system in a region of a unit cell as part of simulating a physical system of interest, as described above at step 204 of FIG. 2. For convenience, the process 300 will be described as being performed by one or more computing devices located in one or more locations. For example, a quantum computation system, e.g., the system 100 for simulating materials using quantum computation 100 of FIG. 1, appropriately programmed in accordance with this specification, can perform the process 300.

The system defines an initial ground state of the physical system in the region of the unit cell as the ground state of a Hamiltonian for the unit cell (step 302). For example, a Hamiltonian H_{sys} describing the physical system may be associated with a ground state of the physical system ψ_{sys}, and a Hamiltonian H_{cell} describing one of the unit cells that constitute the physical system may be associated with a ground state ψ_{cell} of the unit cell. Using this notation, the system may define an initial ground state ψ⁽⁰⁾ of the physical system in the region of the unit cell by

ψ⁽⁰⁾=ψ_{cell}.

The system iteratively processes the initial ground state and subsequent ground states until completion of an event occurs, wherein for each iteration a quantum computation is performed (step 304). As described above with reference to FIG. 2, in some implementations each of the multiple unit cells that constitute the physical system interact with other unit cells in the physical system, e.g., with respective neighboring unit cells. Due to these interaction, the ground state ψ_{cell} of a unit cell may not provide any meaningful information about the ground state ψ_{sys} of the physical system as a whole. However, due to the structure of the physical system, e.g., the regularity and symmetry described above with reference to FIG. 2, meaningful information about the ground state ψ_{sys} of the physical system as a whole can be determined from the ground state ψ_{sys} of the physical system in a local region around a unit cell.

Therefore, the system iteratively processes the initial ground state and subsequent ground states until a completion event occurs to determine an approximated ground state of the physical system in a region of a unit cell. In some implementations the completion of the event occurs when a processed ground state for the iteration converges with a processed ground state for the previous iteration, e.g., when a processed ground state for the iteration is within a predetermined distance in Hilbert space to the processed ground state for the previous iteration. An example iteration of processing a ground state ψ^{(j)} to approximate a ground state of a physical system in a region of a unit cell is described in detail below with reference to FIG. 4.

FIG. 4 is a flow diagram of an example iteration 400 of processing a ground state to approximate a ground state of a physical system in a region of a unit cell. For example, the process 400 may describe a j^{th} iteration of processing an initial or subsequent ground state until a completion event occurs as described above at step 204 of FIG. 2. For convenience, the process 400 will be described as being performed by one or more computing devices located in one or more locations. For example, a quantum computation system, e.g., the system 100 for simulating materials using quantum computation 100 of FIG. 1, appropriately programmed in accordance with this specification, can perform the process 400.

The system determines an embedding Hamiltonian for the iteration (step 402). An embedding Hamiltonian H_{emb} is a Hamiltonian whose ground state ψ_{emb} is statistically close to the ground state of ψ_{sys} in the region of the unit cell. The Hamiltonian H_{emb} may not be larger than the Hamiltonian H_{cell} describing the unit cell. The system may determine a Hamiltonian H_{emb}^{j} for the iteration by performing a classical computation.

In some implementations performing a classical computation may include applying Density Matrix Embedding Theory (DMET) to determine the embedding Hamiltonian for the iteration. Applying DMET to determine an embedding Hamiltonian may include applying an embedding algorithm subroutine A that takes a ground state for the previous iteration ψ_{emb}^{(j-1)} as input and produces a corresponding embedded Hamiltonian H_{emb}^{j} as output, that is

A(ψ^{(j-1)})=H_{emb}^{j}.

The system performs a quantum computation to determine a ground state ψ_{emb}^{(j)} of the embedding Hamiltonian H_{emb}^{j} for the iteration (step 404). In some implementations performing the quantum computation to determine the ground state of the embedding Hamiltonian for the iteration may include performing a variational method. Variational methods can be used to determine eigenstates, e.g., the ground state, of a given quantum system. For example, variational methods may be used to determine a quantum state |ψ [symbol] of a quantum system which is a lowest energy eigenstate of a Hamiltonian H so that H|ψ [symbol] =E₀|ψ [symbol] . For example, some variational methods may approximately prepare |ψ [symbol] by parameterizing a guess wavefunction |ϕ(\vec{θ}) [symbol] , known as an ansatz, in terms of a polynomial number of parameters denoted by the vector \hat{θ}. The quantum variational principle then holds that

$$\frac{〈 φ ( \vec{θ} ) \vert H \vert φ ( \vec{θ} ) 〉}{〈 φ ( \vec{θ} ) \vert φ ( \vec{θ} ) 〉} \geq {E}_{0} ,$$

with equality when |ϕ(\vec{θ}) [symbol] =|ψ [symbol] . Accordingly, |ψ [symbol] may be approximated with |ϕ(\vec{θ}) [symbol] by solving for \vec{θ} which makes the above inequality as tight as possible within the parameterization.

In some implementations the variational method comprises a variational quantum eigensolver (VQE) procedure. A VQE procedure parameterizes |ϕ(\vec{θ}) [symbol] by the action of a parameterized quantum circuit U(\vec{θ}) on an initial state |ϕ [symbol] , i.e.,

|ϕ(\vec{θ}) [symbol] ≡U(\vec{θ})|ϕ [symbol] .

The initial state |ϕ [symbol] may be a quantum state that is trivial to prepare with a quantum circuit, e.g., a product state in the standard basis. Conversely, the parameterized state |ϕ(\vec{θ}) [symbol] may be a quantum state that is very complicated to prepare. For example, the parameterized state |ϕ(\vec{θ}) [symbol] can be a quantum state spanning an exponential number of basis states in the standard basis and thus cannot be represented on any classical computer, e.g., due to memory limitations, even when the unitary operator U is relatively shallow. The mapping U(\vec{θ}) may be represented as a concatenation of parameterized quantum gates, e.g.,

U(\vec{θ})≡U₁(θ₁)U₂(θ₂) . . . Uₙ(θₙ)

where each Uᵢ(θᵢ) represents a quantum circuit element that is decomposed into universal quantum gates and \vec{θ} represents n scalar values {θᵢ}.

After parameterizing |ϕ(\vec{θ}) [symbol] accordingly, the VQE procedure performs a quantum computation. The VQE procedure uses quantum hardware to measure an expectation value of the Hamiltonian H with respect to the parameterized quantum state |ϕ(\vec{θ}) [symbol] . To do this, the VQE procedure repeatedly prepares copies of the quantum state |ϕ(\vec{θ}) [symbol] and performs repeated measurements of local Hamiltonian terms that define H. For example, generally any Hamiltonian H may be decomposed into a sum of terms

$$H = {\sum }_{γ = 1}^{L} {a}_{γ} {H}_{γ} ,$$

where a_{γ} represents real-valued scalars and each H_{γ} represents a Hamiltonian, e.g., a 1-sparse Hamiltonian that can be easily measured. It is noted that this decomposition is always possible in such a way that L is at most polynomially large. Accordingly, the VQE procedure measures each term in the above expression to obtain the expectation value of |ϕ(\vec{θ}) [symbol] , as given below

$$〈 H 〉 ( \vec{θ} ) = {\sum }_{γ = 1}^{L} {a}_{γ} 〈 φ ( \vec{θ} ) \vert {H}_{γ} \vert φ ( \vec{θ} ) 〉 .$$

The final step of the VQE procedure includes minimizing the quantity [symbol] H [symbol] (\vec{θ}) to suggest a new set of parameters \vec{θ}. In some implementations minimizing the quantity [symbol] H [symbol] (\vec{θ}) may be performed using a classical computer. In such a case, example methods used include gradient-free greedy methods, e.g., Powell's method or Nelder-Mead. The VQE procedure may be iterated until the value of [symbol] H [symbol] (\vec{θ}) converges, upon which properties of the quantum state |ϕ(\vec{θ}) [symbol] may be probed experimentally.

As described above, performing the variational method may include performing one or more quantum computations and one or more classical computations. For example, as described with reference to the VQE procedure, the method may include preparing a parameterized quantum state and measuring an expectation value of the embedding Hamiltonian with respect to the parameterized quantum state using quantum hardware. The measurement results may be provided to a classical computer that performs a minimization of the energy landscape to determine updated values of quantum state parameters that, upon convergence, describe the ground state of the embedding Hamiltonian for the iteration.

The system determines whether the completion event occurs (step 406). For example, as described above with reference to step 304 of FIG. 3, in some implementations the completion of the event occurs when a processed ground state for the iteration converges with a processed ground state for the previous iteration.

In response to determining that the completion event has not occurred, the system provides the determined ground state of the embedding Hamiltonian for the iteration as a subsequent state (step 408a). The system may then repeat steps 402-406 until it is determined that the completion event has occurred.

In response to determining that the completion event has occurred, the system defines the determined ground state of the embedding Hamiltonian as an approximated ground state of the physical system in the region of the unit cell (step 408b). As described above with reference to FIG. 2, the approximated ground state of the physical system in the region of the unit cell describes properties of the physical system as a whole. Therefore, the system may define the determined ground state of the embedding Hamiltonian as an approximated ground state of the physical system in the region of the unit cell and use the approximated ground state to simulate the physical system.

Implementations of the digital and/or quantum subject matter and the digital functional operations and quantum operations described in this specification can be implemented in digital electronic circuitry, suitable quantum circuitry or, more generally, quantum computational systems, in tangibly-embodied digital and/or quantum computer software or firmware, in digital and/or quantum computer hardware, including the structures disclosed in this specification and their structural equivalents, or in combinations of one or more of them. The term “quantum computational systems” may include, but is not limited to, quantum computers, quantum information processing systems, quantum cryptography systems, or quantum simulators.

Implementations of the digital and/or quantum subject matter described in this specification can be implemented as one or more digital and/or quantum computer programs, i.e., one or more modules of digital and/or quantum computer program instructions encoded on a tangible non-transitory storage medium for execution by, or to control the operation of, data processing apparatus. The digital and/or quantum computer storage medium can be a machine-readable storage device, a machine-readable storage substrate, a random or serial access memory device, one or more qubits, or a combination of one or more of them. Alternatively or in addition, the program instructions can be encoded on an artificially-generated propagated signal that is capable of encoding digital and/or quantum information, e.g., a machine-generated electrical, optical, or electromagnetic signal, that is generated to encode digital and/or quantum information for transmission to suitable receiver apparatus for execution by a data processing apparatus.

The terms quantum information and quantum data refer to information or data that is carried by, held or stored in quantum systems, where the smallest non-trivial system is a qubit, i.e., a system that defines the unit of quantum information. It is understood that the term “qubit” encompasses all quantum systems that may be suitably approximated as a two-level system in the corresponding context. Such quantum systems may include multi-level systems, e.g., with two or more levels. By way of example, such systems can include atoms, electrons, photons, ions or superconducting qubits. In many implementations the computational basis states are identified with the ground and first excited states, however it is understood that other setups where the computational states are identified with higher level excited states are possible. The term “data processing apparatus” refers to digital and/or quantum data processing hardware and encompasses all kinds of apparatus, devices, and machines for processing digital and/or quantum data, including by way of example a programmable digital processor, a programmable quantum processor, a digital computer, a quantum computer, multiple digital and quantum processors or computers, and combinations thereof. The apparatus can also be, or further include, special purpose logic circuitry, e.g., an FPGA (field programmable gate array), an ASIC (application-specific integrated circuit), or a quantum simulator, i.e., a quantum data processing apparatus that is designed to simulate or produce information about a specific quantum system. In particular, a quantum simulator is a special purpose quantum computer that does not have the capability to perform universal quantum computation. The apparatus can optionally include, in addition to hardware, code that creates an execution environment for digital and/or quantum computer programs, e.g., code that constitutes processor firmware, a protocol stack, a database management system, an operating system, or a combination of one or more of them.

A digital computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a digital computing environment. A quantum computer program, which may also be referred to or described as a program, software, a software application, a module, a software module, a script, or code, can be written in any form of programming language, including compiled or interpreted languages, or declarative or procedural languages, and translated into a suitable quantum programming language, or can be written in a quantum programming language, e.g., QCL or Quipper.

A digital and/or quantum computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A digital and/or quantum computer program can be deployed to be executed on one digital or one quantum computer or on multiple digital and/or quantum computers that are located at one site or distributed across multiple sites and interconnected by a digital and/or quantum data communication network. A quantum data communication network is understood to be a network that may transmit quantum data using quantum systems, e.g. qubits. Generally, a digital data communication network cannot transmit quantum data, however a quantum data communication network may transmit both quantum data and digital data.

The processes and logic flows described in this specification can be performed by one or more programmable digital and/or quantum computers, operating with one or more digital and/or quantum processors, as appropriate, executing one or more digital and/or quantum computer programs to perform functions by operating on input digital and quantum data and generating output. The processes and logic flows can also be performed by, and apparatus can also be implemented as, special purpose logic circuitry, e.g., an FPGA or an ASIC, or a quantum simulator, or by a combination of special purpose logic circuitry or quantum simulators and one or more programmed digital and/or quantum computers.

For a system of one or more digital and/or quantum computers to be “configured to” perform particular operations or actions means that the system has installed on it software, firmware, hardware, or a combination of them that in operation cause the system to perform the operations or actions. For one or more digital and/or quantum computer programs to be configured to perform particular operations or actions means that the one or more programs include instructions that, when executed by digital and/or quantum data processing apparatus, cause the apparatus to perform the operations or actions. A quantum computer may receive instructions from a digital computer that, when executed by the quantum computing apparatus, cause the apparatus to perform the operations or actions.

Digital and/or quantum computers suitable for the execution of a digital and/or quantum computer program can be based on general or special purpose digital and/or quantum processors or both, or any other kind of central digital and/or quantum processing unit. Generally, a central digital and/or quantum processing unit will receive instructions and digital and/or quantum data from a read-only memory, a random access memory, or quantum systems suitable for transmitting quantum data, e.g. photons, or combinations thereof.

The essential elements of a digital and/or quantum computer are a central processing unit for performing or executing instructions and one or more memory devices for storing instructions and digital and/or quantum data. The central processing unit and the memory can be supplemented by, or incorporated in, special purpose logic circuitry or quantum simulators. Generally, a digital and/or quantum computer will also include, or be operatively coupled to receive digital and/or quantum data from or transfer digital and/or quantum data to, or both, one or more mass storage devices for storing digital and/or quantum data, e.g., magnetic, magneto-optical disks, optical disks, or quantum systems suitable for storing quantum information. However, a digital and/or quantum computer need not have such devices.

Digital and/or quantum computer-readable media suitable for storing digital and/or quantum computer program instructions and digital and/or quantum data include all forms of non-volatile digital and/or quantum memory, media and memory devices, including by way of example semiconductor memory devices, e.g., EPROM, EEPROM, and flash memory devices; magnetic disks, e.g., internal hard disks or removable disks; magneto-optical disks; CD-ROM and DVD-ROM disks; and quantum systems, e.g., trapped atoms or electrons. It is understood that quantum memories are devices that can store quantum data for a long time with high fidelity and efficiency, e.g., light-matter interfaces where light is used for transmission and matter for storing and preserving the quantum features of quantum data such as superposition or quantum coherence.

Control of the various systems described in this specification, or portions of them, can be implemented in a digital and/or quantum computer program product that includes instructions that are stored on one or more non-transitory machine-readable storage media, and that are executable on one or more digital and/or quantum processing devices. The systems described in this specification, or portions of them, can each be implemented as an apparatus, method, or system that may include one or more digital and/or quantum processing devices and memory to store executable instructions to perform the operations described in this specification.

While this specification contains many specific implementation details, these should not be construed as limitations on the scope of what may be claimed, but rather as descriptions of features that may be specific to particular implementations. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the implementations described above should not be understood as requiring such separation in all implementations, and it should be understood that the described program components and systems can generally be integrated together in a single software product or packaged into multiple software products.

Particular implementations of the subject matter have been described. Other implementations are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method comprising:
determining a physical system of interest, wherein the physical system comprises a plurality of unit cells;
performing a quantum computation to approximate a ground state of the physical system in a region of one of the unit cells; and
providing the approximated ground state of the physical system in the region of the unit cell as output.

2. The method of claim 1, wherein the quantum computation to approximate the ground state of the physical system in the region of the unit cell comprises:
defining an initial ground state of the physical system in the region of the unit cell as the ground state of a Hamiltonian for the unit cell; and
iteratively processing the initial ground state and subsequent ground states until completion of an event occurs, wherein for each iteration a quantum computation is performed.

3. The method of claim 2, wherein the processing comprises for each iteration:
determining an embedding Hamiltonian for the iteration;
performing a quantum computation to determine a ground state of the embedding Hamiltonian for the iteration;
determining whether the completion event occurs;
in response to determining that the completion event has not occurred, providing the determined ground state of the embedding Hamiltonian for the iteration as a subsequent state; and
in response to determining that the completion event has occurred, defining the determined ground state of the embedding Hamiltonian as an approximated ground state of the physical system in the region of the unit cell.

4. The method of claim 3, wherein determining an embedding Hamiltonian for the iteration comprises performing a classical computation.

5. The method of claim 4, wherein performing the classical computation comprises applying Density Matrix Embedding Theory (DMET).

6. The method of claim 3, wherein performing the quantum computation to determine the ground state of the embedding Hamiltonian for the iteration comprises performing a variational method.

7. The method of claim 6, wherein the variational method comprises a variational quantum eigensolver.

8. The method of claim 6, wherein performing the variational method comprises performing one or more quantum computations and one or more classical computations.

9. The method of claim 2, wherein the completion of the event occurs when a processed ground state for the iteration converges with a processed ground state for the previous iteration.

10. The method of claim 1, wherein the approximated ground state of the physical system in the region of the unit cell describes properties of the whole physical system.

11. The method of claim 1, wherein a unit cell defines a symmetry and structure of the physical system.

12. The method of claim 1, wherein the physical system is a material.

13. The method of claim 12, further comprising using the outputted ground state of the physical system in the region of the unit cell to simulate properties of the material.

14. The method of claim 1, further comprising using the outputted ground state of the physical system in the region of the unit cell to determine properties of the physical system.

15. An apparatus comprising:
quantum hardware;
one or more classical processors;
wherein the apparatus is configured to perform operations comprising:
determining a physical system of interest, wherein the physical system comprises a plurality of unit cells;
performing a quantum computation to approximate a ground state of the physical system in a region of one of the unit cells; and
providing the approximated ground state of the physical system in the region of the unit cell as output.

16. The apparatus of claim 15, wherein the quantum computation to approximate the ground state of the physical system in the region of the unit cell comprises:
defining an initial ground state of the physical system in the region of the unit cell as the ground state of a Hamiltonian for the unit cell; and
iteratively processing the initial ground state and subsequent ground states until completion of an event occurs, wherein for each iteration a quantum computation is performed.

17. The apparatus of claim 16, wherein the processing comprises for each iteration:
determining an embedding Hamiltonian for the iteration;
performing a quantum computation to determine a ground state of the embedding Hamiltonian for the iteration;
determining whether the completion event occurs;
in response to determining that the completion event has not occurred, providing the determined ground state of the embedding Hamiltonian for the iteration as a subsequent state; and
in response to determining that the completion event has occurred, defining the determined ground state of the embedding Hamiltonian as an approximated ground state of the physical system in the region of the unit cell.

18. The apparatus of claim 15, wherein the quantum hardware comprises one or more qubits.

19. The apparatus of claim 18, wherein the one or more qubits comprise superconducting qubits.

20. The apparatus of claim 15, wherein the quantum hardware comprises a quantum circuit.

21. The apparatus of claim 20, wherein the quantum circuit comprises one or more quantum logic gates.
